(19) United States
(12) Patent Application Publication
(10) Pub. No.: US 2024/0316280 A1
(43) Pub. Date: Sep. 26, 2024

(54) RING FOR ASPIRATING A SYRINGE

(71) Applicant: Andrew Saurin, Oceanside, CA (US)

(72) Inventor: Andrew Saurin, Oceanside, CA (US)

(21) Appl. No.: 18/602,315

(22) Filed: Mar. 12, 2024

Related U.S. Application Data
(60) Provisional application No. 63/453,446, filed on Mar. 20, 2023.

(51) Int. Cl. A61M 5/31

(52) U.S. Cl. CPC A61M 5/3148; A61M 2205/586; A61M 2209/088; A61M 2210/083

(57) ABSTRACT

A ring for facilitated aspiration of a syringe is provided. The ring is preferably worn on the thumb of a user, but may be used on other fingers. An opening slot is located along the perimeter of the ring. The opening slot has a curved passageway, formed by the first end and the second end of the ring being curved. The opening slot secures a portion of a syringe. A v-shaped slit on the second end of the ring receives and secures various sized syringes.

### REFERENCE TO RELATED APPLICATIONS

This application claims priority to U.S. Provisional Application No. 63/453,446 which was filed on Mar. 20, 2023, the entire contents of which are incorporated by reference herein.

### BACKGROUND OF THE INVENTION

A ring for facilitated aspiration of a syringe is provided. The ring is preferably worn on the thumb of a user, but may be used on other fingers. An opening slot is located along the perimeter of the ring. The opening slot has a curved passageway, formed by the first end and the second end of the ring being curved. The opening slot secures a portion of a syringe. A v-shaped slit on the second end of the ring receives and secures various sized syringes.

Rings for securing syringes are known. For example, U.S. Pat. No. 11,534,552 to Kleyman discloses a syringe finger ring and finger ring inserts, that in various embodiments, provides or connects to a syringe that comprises a syringe body capable of having a volume of material therein and a plunger disposed to enter the syringe body and dispense material from the syringe body by movement of plunger, and a novel a finger ring and related structures having an open area and connected, by various structures, to the plunger adapted to receive a finger within the finger ring open area, including a spacer of various embodiments inserted within the open area to selectively reduce the open area and provide more controllable engagement of the syringe plunger by the operator finger in distal and proximal strokes.

Still further, U.S. Patent Publication No.: 20150238698 to Perry discloses a device for facilitating an aspiration procedure during a dermal filler injection treatment. The device can be connected to a dermal filler syringe plunger, and includes a thumb stop element for engaging a physician's thumb.

However, these patent documents fail to describe a ring for aspirating a syringe which is easy to use. Further, these patents fail to provide for a ring for aspirating a syringe which allows for the proper securing and control of the syringe.

### SUMMARY OF THE INVENTION

A ring for facilitated aspiration of a syringe is provided. The ring is preferably worn on the thumb of a user, but may be used on other fingers. An opening slot is located along the perimeter of the ring. The opening slot has a curved passageway, formed by the first end and the second end of the ring being curved. The opening slot secures a portion of a syringe. A v-shaped slit on the second end of the ring receives and secures various sized syringes.

An advantage of the present ring is that the present ring provides for easy aspiration of a syringe, which is critical in medical procedures.

An advantage of the present ring for aspirating a syringe is that the present ring may be used with a large variety of syringes.

Another advantage of the present ring for aspirating a syringe is that the present ring does not require an adapter.

Yet another advantage of the present ring for aspirating a syringe is that the present ring is durable.

Still another advantage of the present ring is that the present ring allows for enhanced control during the aspiration process.

Yet another advantage of the present ring is that the present ring provides for improved grip, stability and precision when aspirating a syringe.

And another advantage of the present ring is that the present ring allows for a more accurate and consistent needle placement during the aspiration and subsequent injection.

And yet another advantage of the present ring is that the present ring may be slightly bendable so as to allow a syringe to be easily grasped by the ring.

For a more complete understanding of the above listed features and advantages of the ring for aspirating a syringe reference should be made to the detailed description and the drawings. Further, additional features and advantages of the invention are described in, and will be apparent from, the detailed description of the preferred embodiments.

### BRIEF DESCRIPTION OF THE DRAWINGS

FIG. 1 illustrates a perspective view of the ring in one embodiment.

FIG. 2 illustrates a top view of the ring in one embodiment.

FIG. 3 illustrates a perspective view of the ring with a syringe inserted.

FIG. 4 illustrates a top view of the ring with a syringe inserted.

FIG. 5 illustrates a view of the v-shaped slit of the second end of the ring in one embodiment.

FIG. 6 illustrates a perspective view of a slightly alternatively, but preferred configuration of the ring.

FIG. 7 illustrates a top view of the ring of FIG. 6.

FIG. 8 illustrates a front view of the ring of FIG. 6.

FIG. 9 illustrates a back view of the ring of FIG. 6.

FIG. 10 illustrates a perspective view of the ring of FIG. 6 with the outline of a syringe inserted into the ring.

### DETAILED DESCRIPTION OF THE PREFERRED EMBODIMENTS

A ring for facilitated aspiration of a syringe is provided. The ring is preferably worn on the thumb of a user, but may be used on other fingers. An opening slot is located along the perimeter of the ring. The opening slot has a curved passageway, formed by the first end and the second end of the ring being curved. The opening slot secures a portion of a syringe. A v-shaped slit on the second end of the ring receives and secures various sized syringes.

The present ring allows for more accurate and consistent needle placement during the aspiration and subsequent injection of a patient. In particular, prior aspiration rings generally result in more undesirable movement of the needle during the aspiration process. Further, prior aspiration rings often require resting the tip of the needle on bone to maintain the same location of the needle during the aspiration to check for possible intravascular placement of the needle prior to injecting. Intravascular injection of many drugs and vaccines is contraindicated and can lead to necrosis and even death.

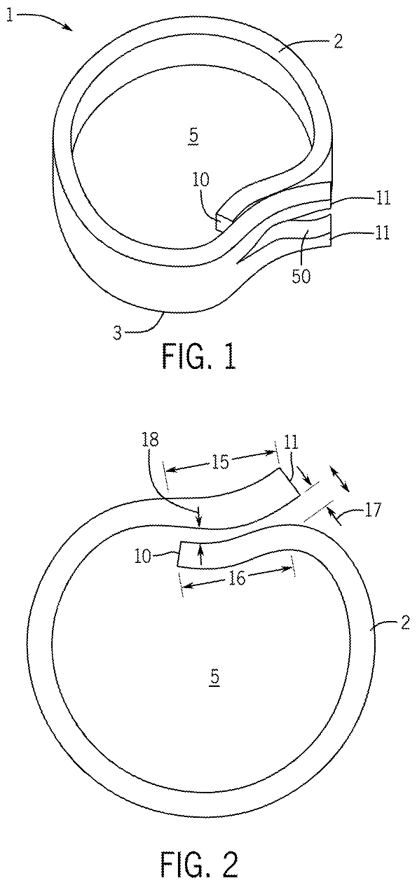

Referring first to FIG. 1, a ring 1 for aspirating a syringe 100 is provided. The ring 1 is preferably made of a durable material, such as stainless steel, brass or silver, but may also be made of more cost-effective materials such as plastic. The present ring 1 for aspirating a syringe 100 may also be powdercoated in different finishes including high grip powdercoats having different colors. Further, the present ring 1 may go through passivation finishing to produce an autoclavable surgical stainless steel to meet sterile medical standards.

In one embodiment, the ring 1 is slightly bendable/flexible so as to better grasp and secure the syringe 100. Prior art aspiration rings may be stretched to accommodate larger syringes after a smaller syringe is used, but these prior aspiration rings generally to not have spring memory which allows them to be used on a small syringe after a larger syringe is used. The present aspiration ring is flexible and may be used going from a smaller syringe to a larger syringe or from a larger syringe to a smaller one repeatedly.

In an embodiment, the ring 1 has a top 2, a bottom 3 and a (generally circular in one embodiment) opening 5 for receiving the thumb or finger of the user. The ring 1 also has a first end 10 and a second end 11 wherein the first end 10 and the second end 11 overlap and wherein the perimeter of the ring is generally circular in one embodiment (the ring of FIGS. 1-4). In an embodiment, the first end 10 is not split, whereas the second end 11 is split by a v-shaped opening 50 (as discussed below). In an embodiment, an inward curved portion 15 of the perimeter of the ring 1 is located at the second end 11 of the ring 1. The inward curved portion 15 of the second end 11 of the perimeter of the ring 1 is ideally one-fifth of the total perimeter of the ring 1; but preferably between one-fourth of the ring and one-six of the total perimeter of the ring 1.

An inward curved portion 16 of first end 10 of the ring 1 may also be present. In one embodiment (the embodiment of FIGS. 1-4), the inward curved portion 15 of the second end 11 of the ring 1 generally similar or identical to the inward curved portion 16 of the first end 10 of the ring 1. The inward curved portions 15, 16 are located directly next to each other, creating a curved passageway 17 (or “gap”) between the two portions 15, 16 of the ring 1. The width 18 of the passageway 17 is great enough to receive the thumb plunger 125 of a syringe 100 as described below.

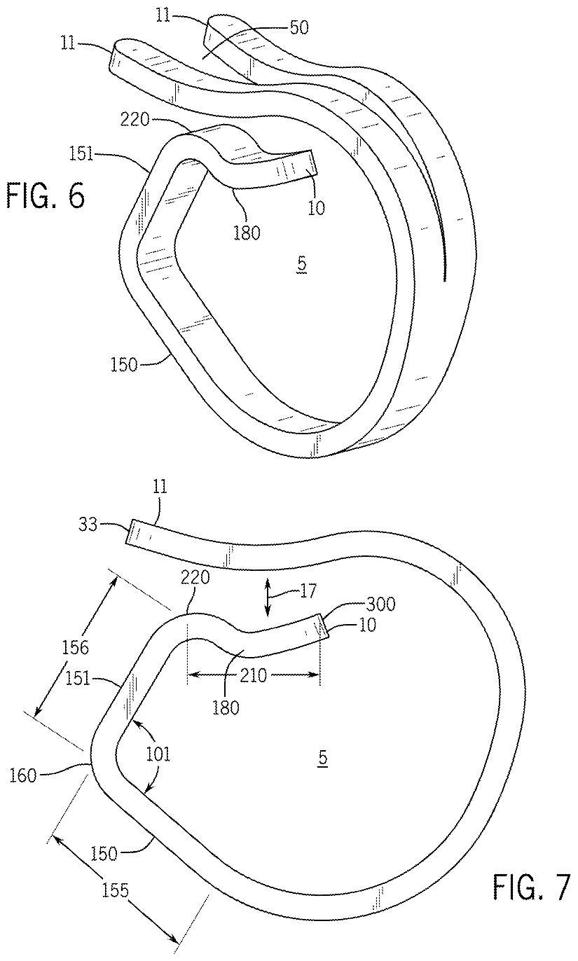

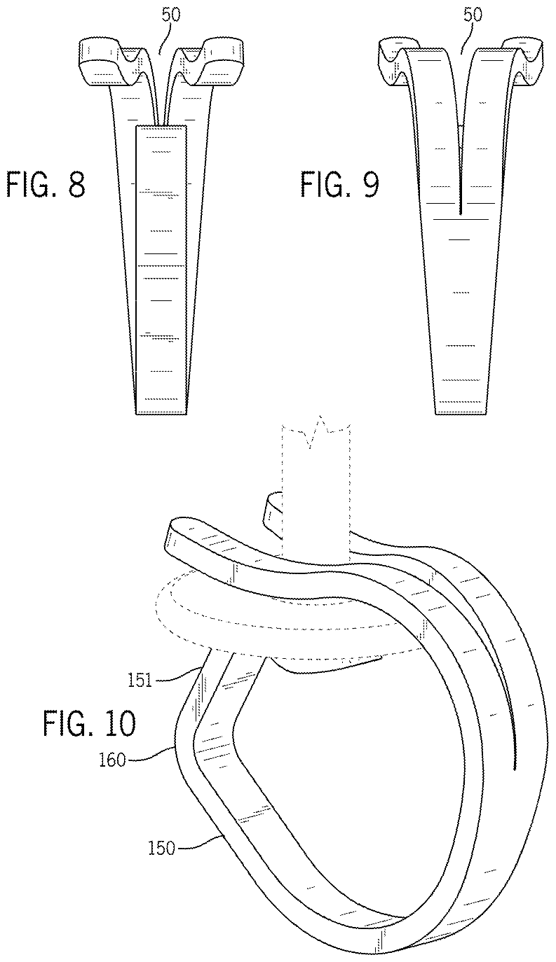

In an embodiment as stated above, the first end 10 and the second end 11 of the ring 1 may have a curved (arc) portion 15, 16. The curves 15, 16 may have an arc that curves in the opposite direction of the curve (arc) of the remainder of the perimeter of the ring 1. More specifically, the ring 1 may have a generally circular outward perimeter with the exception of a curved arc portion 15 of the perimeter being indented inward (at the second end 11 of the ring 1); and with the exception of the generally flat portions shown in the alternative and preferred embodiment of FIGS. 6 through 10.

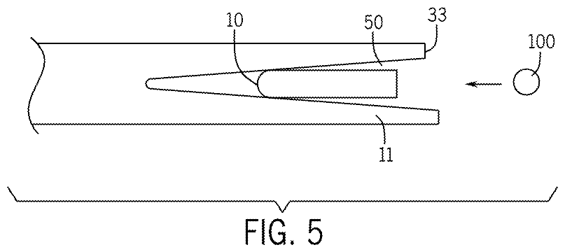

In an embodiment, the second end 11 of the ring 1 may have an elongated v-shaped slit 50 (or “bunny ears” or “triangular slit”) as best illustrated in FIG. 5. The elongated v-shaped slit 50 may decrease in width as it moves away from the terminal end 33 of the second end 11 of the ring 1. The v-shaped slit 50 may receive a portion of the syringe 100. In particular, the thumb plunger 125 of the syringe 100 may be placed between the first end 10 and the second end 11 of the ring 1 (in the passageway 17) so that a portion of the shaft 130 of the syringe 100 passes through the v-shaped slit 50 while the thumb plunger 125 of the syringe 100 remains secured within the passageway 17 between the first end 10 and the second end 11 of the ring 1.

The first end 10 of the ring I may lack an elongated v-shaped slit. The first end 10 of the ring 1 may be located directly beneath the v-shaped slit 50 of the second end 11 such that, when looking down on the ring 1, the first end 10 is visible through the v-shaped slit 50 of the second end 11 (as shown in FIG. 5).

The slit 50 is v-shaped so that the ring 1 is suitable for use in connection with various sized syringes 100 having different diameter shafts 130. In particular, a syringe 100 having a small or large diameter 400 shaft 130 may be used in connection with the present ring 1. The smaller the diameter 400 of the syringe 100, the farther into the v-shaped slit 50 the syringe 100 will be inserted and the farther away from the terminal end 33 of the second end 11 the syringe 100 will be secured.

In an embodiment, the curved passageway 17 located between the inward curved portion 16 and the inward curved portion 15 allows the thumb plunger 125 to be slid in and secured to the ring 1 (due to the curved nature of the passageway 17 compared to the flat plunger 125 of the syringe 100). More specifically, if the passageway 17 were straight, the thumb plunger 125 might accidentally slide out of the ring 1; however, the curved portion of the passageway 17 secures the thumb plunger 125 of the syringe 100 within the passageway 17 by friction. Further, the curved nature of the passageway 17 may slightly bend as well as the passageway 17 may slightly bend the thumb plunger 125 itself so as to grasp and secure the thumb plunger 125 within the passageway 17 by friction.

As a user inserts his/her thumb (or finger) into the opening 5 of the ring 1, the user may move his/her thumb (and therefore the ring 1 itself) and other fingers on that same hand to aspirate the syringe 100 or perform the injection. Because the ring 1 grips and holds the syringe 100 by the friction created by the curvature of the passageway 17, the healthcare provider only needs to use one hand to inject a patient after the ring has been prepared by inserting the syringe 100 into the passageway 17 of the ring 1. As a result, the healthcare worker's other hand is free to perform other tasks. Other aspiration rings require the healthcare provider to use two hands to insert the syringe onto the ring and to insert the syringe into the patient.

While other syringe 100 securing rings 1 may bend to accommodate the thumb or finger of a person, the present ring 1 may bend to accommodate the syringe plunger 125. This results in an easier to use syringe securing ring. Further, a user may elect to insert the syringe 100 into the curved passageway 17 with either the v-shaped opening 50 facing upward (like FIG. 7) or downward (like FIG. 1) depending on the user's preference.

Referring now to FIGS. 6 to 10, the preferred embodiment of the ring 1 is shown. In this preferred embodiment, the ring 1 may also have a first generally flat surface 150 and a second generally flat surface 151 along the perimeter of the ring 1. The first generally flat surface 150 and the second generally flat surface 151 may form an angle 101 between seventy and one hundred degrees; but preferably slightly greater than ninety degrees. Ideally, the first generally flat surface 150 is greater in length 155 than a length 156 of the second generally flat surface 151 so as to increase the spring memory of the ring 1.

The angle 101 of the first generally flat surface 150 to the second generally flat surface 151 may be such that the first end 10 and the second end 11 of the ring may create the optimal spring memory and pressure to secure the thumb plunger 125 of the syringe 100 during use of the ring 1 within the passageway 17 (which slightly increases and decreases in width 18). A bend 160 which forms the angle 101 of the first generally flat surface 150 to the second generally flat surface 151 creates optimal spring memory of the ring 1 which is generally greater than the spring memory of a generally circular embodiment of the ring 1 as shown in FIG. 1. This optimal pressure may prevent the syringe 100 from accidently becoming dislodged from the ring 1 while also allowing the user to remove the syringe 100 easily when desired.

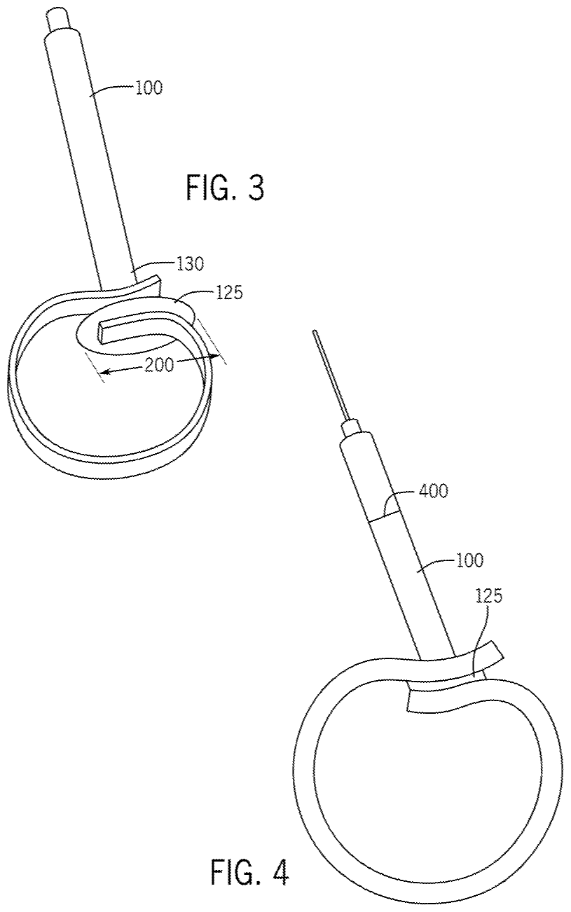

In an embodiment the thumb plunger 125 of the syringe 100 may have a diameter 200 (FIG. 3). In an embodiment, the first end 10 of the ring 1 may have a downward bend 180 (shown in FIG. 7) which creates spring memory. The first end 100 may also have a length 210 defined by a terminal end 300 of the first end 10 and a bump 220 of the first end 10. The length 210 of the first end 10 is ideally less than a diameter 200 of the thumb plunger 125 of the syringe 100 so that both the bump 220 and the terminal end 300 of the first end 10 applies pressure to the thumb plunger 125 of the syringe 100 and secures the thumb plunger 125 within the passageway 17. The first end 10 of the ring 1 may therefore be more curved than the second end 11 of the ring 1 as a result of the first end 10 being more concaved than the second end 11 in an embodiment.

In an embodiment, the first end 10 and the second end 11 of the ring 1 may secure the syringe 100 to the ring 1 without a user needing to actually touch the syringe 100 during the aspiration process. More specifically, during aspiration, the user's thumb or finger need not contact the syringe 100 in order to keep the syringe 100 secured between the first end 10 and the second end 11 of the ring 1. Further, during the aspiration and needle insertion process, the healthcare provider need not even touch the syringe plunger 125 at all. In particular, the healthcare provider may simply press the underside of the first end 10 of the ring 1. This allows the healthcare provider to constantly have a familiar feel when using the ring 1.

Finally, a system may be provided wherein the system includes both a ring 1 and a syringe 100 having the features outlined above. The system is ideal for securing a syringe 100 to a ring 1 for a medical procedure.

Although embodiments of the invention are shown and described therein, it should be understood that various changes and modifications to the presently preferred embodiments will be apparent to those skilled in the art. Such changes and modifications may be made without departing from the spirit and scope of the invention and without diminishing its attendant advantages.

I claim:

1. A ring for a syringe comprising:
a gap passageway between a first end and a second end of the ring wherein the gap passageway secures a plunger of the syringe.

2. The ring for a syringe of claim 1 wherein the gap passageway is curved.

3. The ring for a syringe of claim 1 wherein the ring is flexible.

4. The ring for a syringe of claim 1 wherein the second end of the ring has an extended slit.

5. The ring for a syringe of claim 4 wherein the extended slit of the second end of the ring is v-shaped.

6. The ring for a syringe of claim 5 wherein the v-shaped extended slit of the second end of the ring has a width that decreases.

7. The ring for a syringe of claim 1 wherein the first end of the ring has an arc opposing the arc of a perimeter of the ring.

8. The ring for a syringe of claim 7 wherein the second end of the ring has an arc opposing the arc of a perimeter of the ring.

9. The ring for a syringe of Claim wherein the first end is not split and wherein the second end is split.

10. A ring and syringe system comprising:
providing a ring;
providing a syringe; and
wherein the ring has a passageway between a first end and a second end of the ring and wherein the passageway secures a plunger of the syringe.

11. The system of claim 10 wherein the passageway is curved.

12. The system of claim 10 wherein the ring is flexible.

13. The system of claim 10 wherein the second end of the ring has an extended slit.

14. The system of claim 13 wherein the extended slit of the second end of the ring is v-shaped.

15. The system of claim 14 wherein the v-shaped extended slit of the second end of the ring has a width that decreases.

16. The system of claim 10 wherein the first end of the ring has an arc opposing the arc of a perimeter of the ring.

17. The system of claim 10 wherein the second end of the ring has an arc opposing the arc of a perimeter of the ring.

18. The system of claim 10 wherein the first end of the ring is not split and wherein the second end of the ring is split.
